(19) United States
(12) Patent Application Publication
(10) Pub. No.: US 2007/0254025 A1
(43) Pub. Date: Nov. 1, 2007

(54) ORAL CONTRACEPTIVE AND ACNE MEDICATION COMBINATION AND TREATMENT OF ACNE WITH REDUCED SIDE EFFECTS

(76) Inventor: Peter J. Cronk, Moorestown, NJ (US)

Correspondence Address: DUANE MORRIS, LLP;IP DEPARTMENT, 30 SOUTH 17TH STREET, PHILADELPHIA, PA, 19103-4196, US

(21) Appl. No.: 11/380,446

(22) Filed: Apr. 27, 2006

(51) Int. Cl. A61K 31/57; A61K 31/203; A61K 9/64

(52) U.S. Cl. 424/456; 514/170; 514/559

(57) ABSTRACT

The present invention provides pharmaceutical compositions for the treatment of acne comprising co-administering a therapeutically effective amount of isotretinoin and a contraceptive in a contraceptively effective dosage wherein the contraceptive does not contain 100 wt % progesterone. Methods of treating acne and unit dosage delivery systems are also provided.

### FIELD OF THE INVENTION

The present invention is related to acne treatments and compositions for treating acne.

### BACKGROUND OF THE INVENTION

The treatment of acne can be summarized into established tiers of care, beginning with topical remedies including topical benzoyl peroxide 5-10% and/or topical antibiotics and retinoic acid (Retin-A®), followed by tretinoin, oral antibiotics, hormone-related therapy, and finally, isotretinoin, or isotretinoin (Accutane®, Anmesteem®, Claravis® and Sotret®).

Oral antibiotics, such as tetracycline, erythromycin, taken by mouth, are usually a critical step in the treatment of acne. Antibiotics limit the growth of acne and decrease redness and inflammation. Over time, however, resistance to these drugs can occur. Tetracyclines, including doxycycline and minocycline are the most common antibiotics used in the treatment of acne. Usually these are prescribed once or twice daily and are taken on an empty stomach for improved absorption. These medications often increase one's sensitivity to sun's ultraviolet rays, and rash is a common side effect in those exposed to sunlight while taking the medications. They are obtained only by prescription. Other antibiotics, including erythromycin and bactrim are used less frequently but can work quite well. After good acne control is achieved, the oral antibiotic dosage is usually slowly lowered to the lowest level needed to keep the person acne free. See “The Acne Guide”, www.afraidtoask.com/acne/acneoral, Mar. 6, 2006.

Estrogen at a sufficient dose has been known to reduce sebum production and improve acne. The FDA has approved the triphasic oral birth-control pill (“the Pill”), which has estrogen in combination with progestin, for treatment of moderate acne. So far, there is no role for estrogen therapy in males.

Up to 90% of people with severe acne can be in complete or near complete cure within 12 to 16 weeks of isotretinoin treatment. This medication is generally reserved for those people whose acne is not responding to the above managements or if scarring is occurring. While it is an incredible medication for treating acne, it is a “big gun” and can cause a number of bothersome, serious, and even deadly side effects.

Isotretinoin's effect on acne is on several different levels. It decreases the size of sebaceous glands, it decreases the amount of bacteria that lives in other forms of treatment and who are not candidates for isotretinoin. Isotretinoin is currently the most effective treatment for severe pustulocystic acne that is resistant to conventional therapy. The most significant side effect is teratogenicity (birth defects), therefore isotretinoin should be used cautiously in women of childbearing age, and the drug should not be prescribed in pregnant or nursing women.

According to leading experts, acne vulgaris warranting oral isotretinoin therapy include: nodulocystic or severe inflammatory acne, acne causing severe scarring or pigmentation, acne that relapses or does not respond to conventional therapy, and affects the social existence, in a high profile job or adolescence.

Isotretinoin tablets are generally available in the strength of about 20 milligrams, and are usually taken twice a day with food or milk to get the best results. Typically, patients are started with about 0.5 mg/kg/day and increased to about 1 mg/kg/day, depending on the severity of the acne and tolerability. Capsules or tablets containing about 10-40 mg of isotretinoin per unit dose are standard therapy. See U.S. Pat. No. 4,545,977, col. 2, which is hereby incorporated by reference. The standard cumulative dose for isotretinoin is 120-150 mg/kg/treatment course to prevent relapse. The same dosage guidelines are applicable to repeated courses of isotretinoin. Treatment course lasts for about 4-7 months, depending on daily dose and clinical progress, but some treatments can be as short as two to four weeks with lower cumulative dosages of about 1 mg-150 mg/Kg, preferably about 60 mg/Kg. See U.S. Pat. No. 5,698,593 which is incorporated herein by reference.

Inactive ingredients for isotretinoin capsules include beeswax, butylated hydroxyanisole, edetate disodium, hydrogenated soybean oil flakes, hydrogenated vegetable oil, and soybean oil. Gelatin capsules contain glycerin and parabens (methyl and propyl), with the following dye systems: 10 mg—iron oxide (red) and titanium dioxide; 20 mg—FD&C Red No. 3, FD&C Blue No. 1, and titanium dioxide; 40 mg—FD&C Yellow No. 6, D&C Yellow No. 10, and titanium dioxide.

A number of patients have benefited by combining isotretinoin with diane (e.g., Diane 35®, antiandrogen-estrogen of cyproterone acetate-ethinyl estradiol). Since both these drugs target different mechanisms in acne pathogenesis, they act synergistically. No drug interaction between the two have been reported. See Sheth R. Poonevala V. Isotretinoin: An Indian experience. Indian J Dermatol Venereol Leprol 2001; 67:180-182.

Although diane, or Diane-35®, has many properties in common with estrogen/progestin combination oral contraceptives, it should not be prescribed solely for its contraceptive properties, according to its manufacturer. The Comprehensive Resource for Physicians, Drug and Illness Information, Diane-35® Berlex Canada; Cyproterone Acetate-Ethinyl Estradiol Acne Therapy, www.rxmed.com/b-main/62pharmaceutical/b2.1/monographs/cps-%20monographs, Mar. 6, 2006.

The currently recommended dosage schedule for the treatment of acne with isotretinoin was established sometime prior to September, 1982, when the FDA approved the use of isotretinoin by prescription. The value of this schedule was determined by treating patients with very severe cases of cystic acne who were admitted to experimental protocols testing the efficacy and toxicity of isotretinoin. Because of the demand for this drug prior to release, only the most severely affected patients were admitted into these initial clinical trials.

Currently, patients being treated have much less severe conditions for a variety of reasons. Patients with severe cystic acne who eventually relapse after treatment with isotretinoin still have much less disease than before their initial exposure to the drug, and patients are being treated in earlier stages of the disease. Dermatologists are more familiar with isotretinoin and are more comfortable in prescribing it. It is now commonplace to treat non-cystic acne with isotretinoin if the acne is chronic, antibiotic-resistant, and is associated with scarring. Most of the severe cases of acne have been treated in the past five years and are in long-term remission. Therefore, it is possible and perhaps likely that the currently used schedule is in fact excessive therapy for a portion of acne patients who have less severe, but antibiotic resistant disease.

According to U.S. Pat. No. 5,698,593, acne can be treated by administering to a patient having mild cystic acne or with scarring non-cystic acne a compound selected from the group consisting of isotretinoin and its derivatives, in an amount of approximately 1.5 to 3 mg/kg/day for a period of from about two to about four weeks. The treatment is then stopped, despite any persistent acne. The patients are then observed for three to four months to determine the need for additional treatment. If minimal acne remains, the short course of treatment may be repeated. If moderate or severe acne is present, then the current recommended method of 1 mg/kg/day for five months may be added.

The method of the '593 patent reduces the number of patients who must receive the full five-month course of treatment with isotretinoin or its derivatives, and thus reduces the total dose administered to patients. This, of course, reduces the duration of acute toxicity, the risk of teratogenicity, the risk of chronic radiologic toxicity, and the number of laboratory tests and physicians' office visits required to monitor the therapy. Nevertheless, even one dose of isotretinoin given to a pregnant woman can produce birth defects, so the lowered risk of teratogenicity may not be low enough.

Starting Mar. 1, 2006, to receive isotretinoin, a patient, her physician and her pharmacy must be registered in the iPLEDGE program. She may only receive up to a 30-day supply of isotretinoin at one time, and she will need a new prescription for each refill. Her prescription must be filled within 7 days of her doctor's office visit. While the iPLEDGE program is promising it can take up to an hour for a physician, patient and parent to fill out the required survey. With increased economic pressures exerted upon physicians' offices by managed care programs, the hour or so needed for each Accutane®-treated patient is a heavy burden. Additionally, a substantial problem exists when dealing with teen-age girls or single women who may deny they are sexually active, since physicians are reluctant to disbelieve their patients.

Accordingly, there is a need to overcome the risk of birth defects associated with isotretinoin.

### SUMMARY OF THE INVENTION

The present invention provides a pharmaceutical composition for the treatment of acne, comprising co-administering a therapeutically effective amount of isotretinoin and a contraceptive in a contraceptively effective daily or bi-daily dosage, wherein the contraceptive does not contain 100 wt % progesterone.

The present invention is designed to counter the teratogenicity associated with current isotretinoin therapy. Because the pharmaceutical composition of this invention contains both isotretinoin and a contraceptive together in a daily or bi-daily dosage, the patients taking this therapy, and especially those that take an oral contraceptive for 28-56 days beforehand, are substantially prevented from conceiving. This treatment option will limit the risk of birth defects caused by patents taking isotretinoin alone, without birth control, or by physicians who prescribe isotretinoin to patients who may not be admitting that they are sexually active.

The compositions of this invention potentially provide synergistic acne therapies, since estrogen at a sufficient dose, has been known to reduce sebum reduction, and in joining together this composition with isotretinoin in a single dose, the acne can be attacked by multiple chemical and/or physiological pathways. This invention contemplates the use of contraceptives which contain estrogen, estrogen and progestogen, mifepristone (also known as RU486), or a combination thereof. Specifically, the estrogen can be in the form of a tri-phasic oral birth-control formulation, which has estrogen in combination with progestin.

In a further embodiment of the present invention, a method of treating acne comprising co-administering for 21 successive days, to a female of child bearing age, a combination of an oral contraceptive in a contraceptively effective daily dosage is provided. The daily dosage also includes in combination, a therapeutically effective amount of isotretinoin, followed by 4-8 days which are free of contraceptive administration, but which include continued administration of said therapeutically effective amount of isotretinoin.

In a still further embodiment of the present invention, an oral acne medication is provided having 28 separate dosage units adapted for successive daily oral administration, which comprises 21 dosage units containing in admixture with a pharmaceutically acceptable carrier, 20-50 micrograms estrogen and 0.1-3.0 mg/Kg of body weight isotretinoin, or other retinoic acid derivative equivalent, and 7 additional dosage units containing in admixture with a pharmaceutically acceptable carrier, 0.1-3.0 mg/Kg of body weight isotretinoin, or other retinoic acid derivative equivalent.

The disclosed therapies are, generally but not necessarily, used by the patient in conjunction with at least one form (e.g., the Pill) and, preferably, two forms (e.g., the Pill and condom, or diaphragm and spermicide, etc.) for at least 4 weeks prior to initiation of treatment, and for at least 4 weeks after said treatment is terminated.

The oral acne medication of this invention can also be provided in 42 initial bi-daily dosage units of contraceptive and isotretinoin, followed by 14 additional bi-daily dosage units of isotretinoin. This will permit the isotretinoin to be taken as currently prescribed with meals in the morning and evening. Similarly, tri-daily dosages can be provided with proportionate dosages.

### BRIEF DESCRIPTION OF THE DRAWINGS

The accompanying drawing illustrates a preferred embodiment of the invention, as well as other information pertinent to the disclosure in which:

FIG. 1 is a top plan view of a blister pack containing twenty-one daily dosage units of isotretinoin and contraceptive in combination, and seven daily dosage units of isotretinoin.

### DETAILED DESCRIPTION OF THE INVENTION

Oral isotretinoin (13-cis-retinoic acid or Accutane® or 13-cis vitamin A) is an isomer of all-trans retinoic acid, a metabolite of retinol (vitamin A). The absorption is enhanced when the drug is taken with food. There is no progressive accumulation of the drug in the skin during long term administration. It has a very short half-life compared to etretinate (7-37 hours). It crosses the placenta. It is rapidly eliminated by the liver and kidneys, with the main mechanism of excretion being hepatic clearance. No parent drug is identified in the urine.

Amongst both synthetic (such as etretinate, acitretin, arotinoids) and natural retinoids (such as all-trans retinoic acid and isotretinoin), only isotretinoin exerts its effect on sebum production and therefore on acne. The most likely mechanism by which isotretinoin leads to clinical improvement in acne is by reducing sebaceous gland size (up to 90%) by decreasing proliferation of basal sebocytes, suppressing sebum production and inhibiting sebocyte differentiation. Sebocyte lipid synthesis is reduced by 75% with daily doses as low as 0.1 mg/kg after four weeks. Other mechanisms include anti-inflammatory, anti-bacterial, inhibition of microbial enzyme activity and desquamation of poral occlusion.

The present invention provides a single pharmaceutical composition for the treatment of antibiotic resistant acne comprising a therapeutically effective amount of isotretinoin and a contraceptive, e.g., estrogen-containing or mifepristone-containing contraceptive, in a contraceptively effective daily or bi-daily dosage. The total number of days during which the estrogen, mifepristone, or combination of progesterone and estrogen are administered daily is preferably about 21. These are followed by 4-8 days which are free of estrogen administration to approximate the natural 28-day menstrual cycle of the female. Day one of the cycle is defined as the first day of menstruation and the days are numbered sequentially thereafter until menstruation occurs again. The cycle usually lasts 28 days but it may be slightly longer or shorter. In actual practice, the pills, capsules or tablets might contain nutritional supplements such as, for example, iron supplements, folic acid, calcium, etc. Thus, in a preferred regimen, phase one would commence sometime between day 1 and day 7 of the menstrual cycle and last about 21 days.

The isotretinoin-contraceptive co-administered composition employed in the present invention comprises separate daily or bi-daily dosage units which are adapted for successive daily or bi-daily oral ingestion. The composition consists essentially of, as the first phase, about 21 daily, or 42 bi-daily, dosage units containing in admixture with a pharmaceutically acceptable carrier, a combination of mifepristone-containing or estrogen-containing contraceptive in combination with a therapeutically effective amount of isotretinoin, optionally followed by 4-8 dosage units free of contraceptive. The contraceptive and/or isotretinoin or other retinoic acid derivative daily or bi-daily dosage is preferably kept constant, or ramped up or down, in all phases. For example, patients may be started out with about 0.5 mg/Kg/day of isotretinoin for the first 7-21 days, followed by a dose of about 1.0 mg/Kg/day of isotretinoin for the next 7-21 days.

Any conventional estrogen may be employed as a suitable component in the contraceptive regimen of this invention. The particular regimen employed in a daily dosage should be equal in contraceptive activity in each phase to a daily dosage of about 20-50 μg of estrogen, such as 17 α-ethinylestradiol, or estrogen in combination with progestogen, e.g., progesterone. Monophasic, biphasic and triphasic regimens including estrogen may also be used. See, for example, U.S. Pat. No. 6,214,815, incorporated herein by reference in its entirety. The preferred dosage is one equal to a daily dosage of about 25-35 μg of 17 α-ethinylestradiol. Commercially available oral contraceptives useful for this purpose include Ortho-Novum 1/35 (7/7/7), Triphasil, Lo/Ovral, Tri-Levlin, to name a few.

In addition to 17 α-ethinylestradiol, esters and ethers of 17 α-ethinylestradiol such as, for example, 17 α-ethinylestradiol 3-dimethylamino propionate, 17 α-ethinylestradiol 3-cyclopentyl ether (quienestrol) and 17 α-ethinylestradiol 3-methyl ether (mestranol) may also be employed as the estrogen component. Natural estrogens such as estrone, estrone sulfate, estrone sulfate piperazine salt, estradiol and estriol, and their esters, as well as the synthetic estrogens, may also be employed. A preferred estrogen is 17 α-ethinylestradiol or 17 α-ethinylestradiol 3-methly ether, with or without cyproterone acetate.

The estrogen, mifepristone, or estrogen/progestogen and isotretinoin components are preferably administered together orally in a pharmaceutically acceptable nontoxic carrier, but they can also be administered separately or parenterally. In general, the effective agents are processed, together with the usually additives, vehicles and/or flavor-ameliorating agents normally employed in pharmacies, in accordance with generally accepted pharmaceutical practices. For the preferred oral administration, tablets, dragrees, caplets, gel-caps, soft capsules, capsules, pills, suspensions or solutions are particularly suitable; for parenteral application, oily solutions such as, for example, sesame oil or castor oil solutions which can optionally additionally contain a diluent such as, for example, benzyl benzoate or benzyl alcohol.

In the case of the preferred oral application, the combination-type isotretinoin-contraceptives are preferably packaged in the form of a pharmaceutical kit or package in which the daily dosages are arranged for proper sequential administration. This invention also relates, therefore, to a pharmaceutical unit which contains combination-type medications such as: isotretinoin-estrogen, isotretinoin-mifepristone, or isotretinoin-estrogen-progestogen, for example, in dosage units in a synchronized, fixed sequence, wherein the sequence or arrangement of the dosage units corresponds to the stages of daily administration.

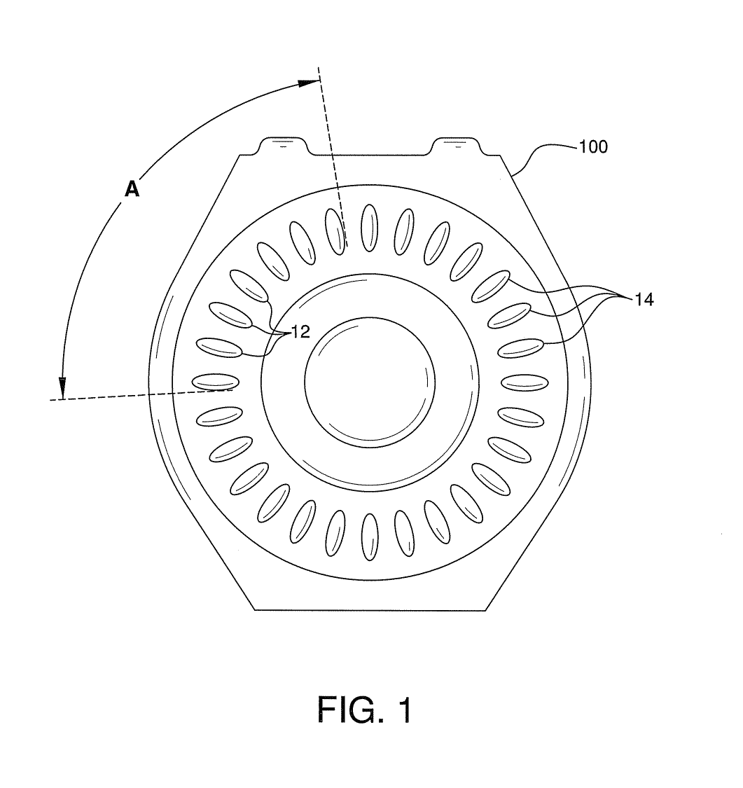

As shown in FIG. 1, the pharmaceutical unit can be, for example, in the form of a transparent package (“blister pack”) having dosage units arranged sequentially and consisting of the tablets 14 for the first phase, for example, 21 dosage units containing in admixture with a pharmaceutically acceptable carrier, 20-50 μg estrogen, more preferably 30-35 μg, and about 0.1-3.0 mg/kg of body weight isotretinoin or other retinoic acid derivative equivalent, most preferably about 0.5-3.0 mg/Kg, followed by the tablets 12 for the second phase, generally shown in region “A”, such as 7 additional dosage units containing 0.1-3.0 mg/cg of body weight isotretinoin or other retinoic acid derivative equivalent, or capsules or tablets of about 20-80 milligrams isotretinoin daily dosage for the average patient. A single capsule or tablet, for example, is to be taken each day over the period of the cycle. In the event a bi-daily co-administration is recommended, such as recommended widely for isotretinoin, the above dosages can be halved to 10-25 μg estrogen-containing contraceptive and 0.05-1.5 mg/kg body weight isotretinoin or other retinoic acid derivative, or alternatively about 10-40 milligrams isotretinoin bi-daily units dosage for the average patient.

Isotretinoin therapy should not be used with progesterone-only birth control hormones (examples: ‘Minipills’ like Aygestin®, Micronor®, Nor-QD® or injectable/implantable products such as Depo-Provera® or Norplant®), since they may not work effectively as a contraceptive, therefore, estrogen, e.g., monophasic and combination estrogen/progesterone, e.g., biphasic or triphasic, contraceptives are recommended. Additionally, although newly acknowledged for its contraceptive activity, mifepristone may have utility.

In a recent study, about 3.56 million British women, approximately a third of those of reproductive age, were determined to be taking the Pill, more than 90 percent of whom are on the combined form that contains estrogen and progesterone, the two female hormones. M. Henderson, “A contraceptive pill that can beat cancer”, The Times Health News, www.timesonline.co.uk/alticle/0,2-2106558,00.html, Mar. 28, 2006. The rest were determined to be taking the mini-Pill, which contains progesterone only. The popularity of the Pill has largely recovered from the 1995 scare that prompted hundreds of thousands to give up oral contraception after “third-generation” Pills that contain different kinds of progesterone were linked to a higher risk of thrombosis.

The combined Pill protects against ovarian and endometrial tumors, but its estrogen content is thought to contribute to a slightly increased risk of breast cancer by some. While the mini-Pill does not have this drawback, it is less effective and has other side-effects, such as heavy bleeding. A new Pill using mifepristone (RU486) is designed to block the action of progesterone, which the body needs to ovulate and support a pregnancy. As it contains no estrogen, it should not promote breast cancer, and by inhibiting progesterone, it is thought that it may even reduce the risk. It is also unlikely to cause other hormonal side-effects, and has the added benefit of stopping periods, which should prevent PMS.

Mifepristone is licensed for use in abortions, though it is used at doses 100 times lower for contraception, e.g., about 2 mg. This invention preferably employs about 0.1-20 mg of mifepristone in combination with isotretinoin, preferably about 1-5 mg, for daily doses, and about 0.05-10 mg, preferably about 0.5-2.5 mg, for bi-daily doses. Mifepristone works by binding to progesterone receptors so that the body cannot respond to the hormone. If given in high doses when a woman is pregnant, it causes miscarriage, but smaller doses can prevent ovulation and conception.

From the foregoing, it can be realized that this invention provides pharmaceutical compositions for the treatment of acne comprising a co-administration of a therapeutically effective amount of isotretinoin and a contraceptive in a contraceptively effective daily or bi-daily dosage, wherein the contraceptive does not contain 100 wt % progesterone. The compositions, methods of treating acne and unit dosage delivery systems of this invention are designed to insure that patients taking isotretinoin are more effectively prevented from conception. The pharmaceutical compositions of this invention can be provided in blister packs containing at least 28 compartments, traditionally used for the delivery of oral contraceptives.

1. A pharmaceutical composition for the treatment of acne comprising a co-administration of a therapeutically effective amount of isotretinoin and a contraceptive in a contraceptively effective dosage, wherein the contraceptive does not contain 100 wt % progesterone.

2. The pharmaceutical composition of claim 1 wherein said composition is in an oral unit dosage form.

3. The pharmaceutical composition of claim 1 wherein said composition comprises from about 0.1 to 3.0 mg/Kg of body weight per day of isotretinoin, and said contraceptive comprising about 20 to 50 μg of an estrogen, or 0.1 mg to 20 mg mifepristone, when provided in a daily dosage form.

4. The pharmaceutical composition of claim 2 wherein said oral unit dosage form is selected from the group consisting of: a capsule, a tablet, a caplet, a gel-cap or soft gelatin capsule.

5. The pharmaceutical composition of claim 4 wherein said composition comprises a contraceptive selected from the group consisting of: estrogen, estrogen and progestogen, mifepristone, or a combination thereof.

6. The pharmaceutical composition of claim 5 wherein said composition includes isotretinoin in an amount of about 0.05-3.0 mg/Kg of body weight per day.

7. The pharmaceutical composition of claim 1 containing two capsules, two tablets, two caplets, two gel-caps or two soft gelatin capsules, each comprising about 0.05 to 1.5 mg/Kg of body weight per day of isotretinoin in combination with a contraceptive comprising about 10 to 25 μg of an estrogen, or 0.05 mg to 10 mg mifepristone, for bi-daily delivery of same.

8. An improved method of treating acne comprising co-administering to a patient in need of such treatment a single composition comprising a therapeutically effective amount of isotretinoin and an estrogen-containing or mifepristone-containing contraceptive in a contraceptively effective, daily or bi-daily dosage.

9. The method of claim 8 further comprising providing said composition in daily or bi-daily dosage form in a blister pack, comprising at least 28 compartments.

10. A method of treating acne comprising co-administering for 21 successive days to a female of child bearing age a combination of an oral contraceptive in a contraceptively effective daily dosage in combination with a therapeutically effective amount of isotretinoin, followed by 4-8 days which are free of contraceptive administration but include continued administration of said therapeutically effective amount of isotretinoin.

11. The method of claim 10 wherein said treatment is continued for about 2 weeks to about 7 months.

12. The method of claim 10 wherein said treatment is continued until said female has received a cumulative dose of isotretinoin of about 1-150 mg/Kg of body weight.

13. The method of claim 10 wherein said oral contraceptive is administered for a period of 28-56 days after isotretinoin therapy has been stopped.

14. The method of claim 10 wherein said oral contraceptive is administered for a period of 28-56 days before isotretinoin therapy initiation.

15. An oral acne medication having 28 separate dosage units adapted for successive daily oral administration comprising 21 dosage units containing in admixture with a pharmaceutically acceptable carrier, 20-50 μg estrogen and 0.1-3.0 mg/Kg of body weight isotretinoin or other retinoic acid derivative equivalent, and 7 additional dosage units containing in admixture with a pharmaceutically acceptable carrier, 0.1-3.0 mg/Kg of body weight isotretinoin or other retinoic acid derivative equivalent.

16. The oral acne medication of claim 15 wherein isotretinoin is provided in 42 initial bi-daily dosage units and 14 additional bi-daily dosage units.

17. A method of treating acne comprising providing:
a) a package of daily dosage units containing isotretinoin or other retinoic acid derivative and separate daily dosage units containing an oral contraceptive, said daily dosage units disposed in a synchronized, fixed sequence, wherein the sequence of dosage units corresponds to the stages of daily administration; and
b) administering said daily dosage units of isotretinoin or other retinoic acid derivative and said separate dosage units of oral contraceptive in a first phase containing at least about 21 days.

18. The method of claim 17 further comprising administering isotretinoin in a second phase of about 4-8 days, which is free of oral contraceptive administration.

19. The method of claim 17 wherein said package comprises a blister pack.

20. The method of claim 17 wherein said isotretinoin is provided in a bi-daily unit dosage form.

21. A pharmaceutical package comprising daily dosage units of an oral contraceptive and daily dosage units of an isotretinoin or other retinoic acid derivative arranged in a synchronized, fixed sequence wherein the sequence of dosage units corresponds to the stages of daily administration.

22. The pharmaceutical package of claim 21 wherein said daily dosage units of said oral contraceptive and said daily dosage units of said isotretinoin or other retinoic acid derivative are administered separately.
